(19) United States
(12) Patent Application Publication
(10) Pub. No.: US 2026/0216385 A1
(43) Pub. Date: Jul. 30, 2026

(54) Antibodies Conjugated With Actinium-225 and Actinium-227, and Related Compositions and Methods

(71) Applicant: Actinium Pharmaceuticals, Inc., New York, NY (US)

(72) Inventors: Dale Ludwig, Rockaway, NJ (US); Steve O'Loughlin, Union City, NJ (US); Vimal Patel, Jersey City, NJ (US); Nitya Ray, East Hanover, NJ (US); Sandesh Seth, New York, NY (US)

(73) Assignee: Actinium Pharmaceuticals, Inc., New York, NY (US)

(21) Appl. No.: 19/299,519

(22) Filed: Aug. 14, 2025

Related U.S. Application Data
(63) Continuation of application No. 17/294,500, filed on Nov. 27, 2019, now abandoned.
(60) Provisional application No. 62/773,234, filed on Nov. 30, 2018.

(51) Int. Cl. A61K 51/10; C07K 16/28

(52) U.S. Cl. CPC A61K 51/1096; A61K 51/1027; C07K 16/2803; C07K 2317/21

(57) ABSTRACT

This invention provides a compositions of matter comprising a therapeutic protein population (such as a HuM195 antibody population) wherein (a) each therapeutic protein in the population is conjugated to one or more actinium atoms, (b) each actinium atom is either ²²⁷Ac or ²²⁵Ac, and (c) the molar ratio of ²²⁷Ac to ²²⁵Ac in the composition is at least 1:1. This invention also provides related synthetic compositions and methods, as well as methods for treating hematologic malignancies.

This application claims the benefit of U.S. Provisional Application No. 62/773,234, filed Nov. 30, 2018, the contents of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to therapeutic protein populations conjugated with ²²⁵Ac and a molar preponderance of ²²⁷Ac.

### BACKGROUND OF THE INVENTION

Radioimmunotherapy is a promising therapeutic strategy for treating cancer. It builds on the proven success of external beam radiation, but in a targeted fashion. Radionuclide particles can emit alpha, beta, and/or gamma radiation during decay, and this radiation can kill cancer cells by causing lethal DNA damage. When linked to a targeted delivery vehicle such as a monoclonal antibody, antibody fragment or other peptide, the energy imparted by the radionuclide warhead can be focused directly on tumor cells following infusion of the radio-conjugate to cancer patients. In the United States, the success of this approach was realized with the regulatory approval of two anti-CD20 radioimmunoconjugate antibodies—Bexxar® and Zevalin®, carrying the beta emitters ¹³¹I (iodine) and ⁹⁰Y (yttrium), respectively—for treating lymphoma.

Further, Lutathera, carrying the beta emitter ¹⁷⁷Lu (lutetium), was approved for treating pancreatic neuroendocrine tumors.

Recently, alpha particle therapy has emerged as a potentially more effective form of targeted radiotherapy for cancer. Unlike beta emitters, alpha emitters release high-energy alpha particles upon decay (identical to the nucleus of a helium-4 atom, which consists of two protons and two neutrons). These particles impart significant linear energy transfer (LET), approximately 100 keV/μm, over a very short path length, typically of only a few cell diameters. The path length of a high-LET alpha particle is so short that the particle cannot pass through a piece of paper. It therefore may be a safer radionuclide for handling and use in therapeutics development. Importantly, alpha particle conjugate therapies can potently kill adjacent antigen-targeted tumor cells, and spare distant normal tissue. As few as one hit to DNA with an alpha particle can generate a lethal double-strand break and kill a tumor cell (Nikula, et al., 1999). Xofigo (²²³RaCl₂) for metastatic prostate cancer is one example of alpha particle radiotherapy.

The high-energy alpha particle-emitting radionuclide Actinium-225 (²²⁵Ac) is a potentially ideal radionuclide for radioimmunotherapy, emitting four high-energy daughter particles over its 10-day half-life. Studies with alpha radio-conjugates have demonstrated that several logs less ²²⁵Ac radioactivity was required to reach LD50 compared to ²¹³Bi, an alpha-emitter with a 46-minute half-life when conjugated to the same antibody. This is presumably due to the longer half-life and greater number of alpha emissions from the ²²⁵Ac radionuclide. Emerging ²²⁵Ac programs targeting CD33 (e.g., ²²⁵Ac-HuM195) for acute myeloid leukemia (Jurcic, 2018), multiple myeloma, myelodysplastic syndrome and PSMA (²²⁵Ac-PSMA-617) are showing promise in clinical studies (Kratochwil, et al., 2017).

The global supply of ²²⁵Ac available for radio-immunoconjugate therapy is currently generated primarily following purification of decay products from a ²²⁹Th source (called a “cow”). This ²²⁹Th cow is, in turn, obtained from ²³³U (uranium) originally produced as a component of the U.S. molten salt breeder reactor program. Total worldwide production is approximately 1.7 Ci/year. The majority of this is generated by the U.S. Department of Energy (Oak Ridge National Laboratory (ORNL) in Oak Ridge, TN and the Institute for Transuranium Elements in Karlsruhe, Germany).

This level of ²²⁵Ac supply is sufficient to meet current clinical demand. However, the amount of ²²⁹Th available for the cow is static, and it is therefore insufficient to meet anticipated commercial needs for ²²⁵Ac supply. For example, upon the successful launch of one or more ²²⁵Ac-based therapies in oncology, demand for this potent radionuclide may require the availability of ²²⁵Ac at levels of as much as 50-150 Ci/year, far greater than can be met with ²²⁹Th cow production.

Alternative production methods for generating ²²⁵Ac are available using particle (e.g. proton) bombardment of a target source, such as ²³²Th or ²²⁶Ra in a linear accelerator (“linac”) or in a cyclotron. Recently, the U.S. DOE (Los Alamos National Lab, Brookhaven National Lab, and Oak Ridge National Lab) has demonstrated the feasibility of producing significant quantities of ²²⁵Ac in a linac through proton bombardment of an immobilized ²³²Th target. Results indicate that as much as 20 Ci of ²²⁵Ac could be produced in a 10-day cycle (Weidner, et al, 2012), and possibly as much as 30 Ci with optimization.

An important issue, though, is the co-purification of ²²⁵Ac with ²²⁷Ac. ²²⁷Ac is a low-energy radionuclide with a long decay half-life of 21.8 years. Purified samples from the linac preparation may contain between 0.2 and 0.7% of ²²⁷Ac, as calculated by specific activity (i.e., radioactivity). Due to the low specific activity of ²²⁷Ac, the calculated molar ratio of ²²⁷Ac to ²²⁵Ac is approximately 5:1 at 0.7% activity. As a result, radiolabeling using DOTA-conjugated linac-produced ²²⁵Ac results in a co-labeling of the target vehicle with both ²²⁵Ac and ²²⁷Ac.

The presence of long-lived ²²⁷Ac is of potential concern, since it can remain in the body for an extended period of time. ²²⁷Ac decays primarily by beta-decay to ²²⁷Th. Radioimmunoconjugates of ²²⁵Ac are typically made by complexation to the chelator DOTA (in the form of p-SCN-Bn-DOTA, as discussed below). DOTA is stably conjugated through linkage to a targeting moiety such as a monoclonal antibody. Theoretical modeling assumes that as much as 70% of the ²²⁷Th decay product from ²²⁷Ac would remain associated with the chelator-antibody, not as free ²²⁷Th, and would therefore retain pharmacokinetic properties of the antibody. Further, this modeling proposes that the absorbed dose contribution of ²²⁷Ac to normal organs is negligible, e.g., <0.7 mGy/MBq to the spleen and <0.1 mGy/MBq to other tissues when modeled using an anti-CD33 antibody such as HuM195 for treating leukemia. In addition, biodistribution studies in rodents comparing free ²²⁵Ac and DOTA-chelated ²²⁵Ac (though not antibody-conjugated ²²⁵Ac) from a ²²⁹Th cow and linac have suggested that the presence of ²²⁷Ac in ²²⁵Ac preparations does not alter the biodistribution of free or chelated ²²⁵Ac in vivo (Dadachova, et al., 2018), and may thus be a suitable replacement for ²²⁹Th-derived ²²⁵Ac for the generation of radioimmunoconjugates.

While the absorbed radiation dose contribution of ²²⁷Ac may be considered negligible in linac-produced ²²⁵Ac, its presence in preparations having roughly five-fold molar excess over ²²⁵Ac would be expected to hinder the efficient labeling of a therapeutic antibody with this material. Calculations for linac-produced ²²⁵Ac with a low-energy ²²⁷Ac impurity profile of 0.7% radioactivity suggest that nearly 85% of the molar mass of purified Ac in the preparation is ²²⁷Ac (see Table 1). While processes for efficient conjugation and labeling of antibodies, fragments or peptides have been demonstrated (Simon, U.S. Pat. No. 9,603,954), it is unknown whether linac-derived ²²⁵Ac would adversely affect molecule labeling, purity and potency. With roughly five times more ²²⁷Ac than ²²⁵Ac present, and despite ²²⁷Ac's low energy, it is unknown whether the free high-energy ²²⁵Ac would be “outcompeted”, thus resulting in poor labeling efficiency. As a result, it is unknown whether the potency of the antibody radio-conjugate would suffer due to the molar excess of conjugated low-energy ²²⁷Ac.

### SUMMARY OF THE INVENTION

This invention provides a first composition of matter comprising a therapeutic protein population wherein (a) each therapeutic protein in the population is conjugated to one or more actinium atoms, (b) each actinium atom is either ²²⁷Ac or ²²⁵Ac, and (c) the molar ratio of ²²⁷Ac to ²²⁵Ac in the composition is at least 1:1.

This invention also provides a second composition of matter comprising a HuM195 antibody population wherein (a) each HuM195 antibody in the population is conjugated to one or more actinium atoms, (b) each conjugated actinium atom is conjugated via p-SCN-Bn-DOTA, (c) each actinium atom is either ²²⁷Ac or ²²⁵Ac, and (d) the molar ratio of ²²⁷Ac to ²²⁵Ac in the composition is between 5:1 and 6:1.

This invention provides a third composition of matter comprising a population of chelated actinium atoms wherein (a) each actinium atom is either ²²⁷Ac or ²²⁵Ac, and (b) the molar ratio of ²²⁷Ac to ²²⁵Ac in the composition is at least 1:1.

This invention further provides a fourth composition of matter comprising a population of chelated actinium atoms wherein (a) each actinium atom is either ²²⁷Ac or ²²⁵Ac, (b) each chelated actinium atom comprises the actinium atom and p-SCN-Bn-DOTA, and (c) the molar ratio of ²²⁷Ac to ²²⁵Ac in the composition is between 5:1 and 6:1.

This invention provides a first synthetic method for making a population of actinium-conjugated therapeutic proteins, comprising contacting, under conjugating conditions, (a) a population of therapeutic proteins and (b) a population of chelated actinium atoms wherein (i) each chelated actinium atom is either ²²⁷Ac or ²²⁵Ac, and (ii) the molar ratio of ²²⁷Ac to ²²⁵Ac in the population of chelated actinium atoms is at least 1:1.

This invention provides a second synthetic method for making a population of actinium-conjugated HuM195 antibodies, comprising contacting, under conjugating conditions, (a) a population of HuM195 antibodies and (b) a population of actinium atoms chelated with p-SCN-Bn-DOTA, wherein (i) each chelated actinium atom is either ²²⁷Ac or ²²⁵Ac, and (ii) the molar ratio of ²²⁷Ac to ²²⁵Ac in the population of chelated actinium atoms is between 5:1 and 6:1.

This invention provides a first therapeutic method for treating a subject, preferably human, afflicted with a hematologic malignancy comprising administering to the subject a therapeutically effective amount of the first pharmaceutical composition, wherein the therapeutic protein is an anti-CD33 antibody.

This invention further provides a second therapeutic method for treating a subject, preferably human, afflicted with acute myeloid leukemia comprising administering to the subject a therapeutically effective amount of the second pharmaceutical composition.

### BRIEF DESCRIPTION OF THE FIGURES

FIG. 1 This figure shows a schematic diagram of the expression plasmids for HuM195. The humanized VL and VH exons of HuM195 are flanked by XbaI sites. The VL exon was inserted into mammalian expression vector pVk, and the VH exon into pVg1 (Co, et al., J. Immunol. 148:1149-1154, 1992).

FIG. 2 This figure shows the complete sequence of the HuM195 light chain gene cloned in pVk between the XbaI and BamHI sites. The nucleotide number indicates its position in the plasmid pVk-HuM195. The VL and CK exons are translated in single letter code; the dot indicates the translation termination codon. The mature light chain begins at the double-underlined aspartic acid (D). The intron sequence is in italics. The polyA signal is underlined.

FIG. 3 This figure shows the complete sequence of the HuM195 heavy chain gene cloned in pVg1 between the XbaI and BamHI sites. The nucleotide number indicates its position in the plasmid pVg1-HuM195. The VH, CH1, H, CH2 and CH3 exons are translated in single letter code; the dot indicates the translation termination codon. The mature heavy chain begins at the double-underlined glutamine (Q). The intron sequences are in italics. The polyA signal is underlined.

FIG. 4 This figure shows the structure of ²²⁵Ac-Lintuzumab (²²⁵Ac-HuM195).

FIG. 5 This figure shows a first flowchart for the production of ²²⁵Ac-HuM195, whereby ²²⁵Ac is first chelated with p-SCN-Bn-DOTA and the resulting chelated complex is bound to HuM195 (lintuzumab) (i.e., a 2-step labeling procedure).

FIG. 6 This figure shows a second flowchart for the production of ²²⁵Ac-HuM195, whereby HuM195 (lintuzumab) is first bound to p-SCN-Bn-DOTA and the resulting antibody is then chelated with ²²⁵Ac (i.e., a 1-step labeling procedure (Simon)).

FIG. 7 This figure shows decay schemes for ²²⁵Ac and ²²⁷Ac (Fassbender, et al.).

FIG. 8 This figure shows the results of two independent labeling comparisons of ²²⁵Ac vs ^{225/7}Ac chelated to HuM195. Preparations 1 and 2 represent two independent conjugation and labeling experiments performed on different dates with different lots of ²²⁵Ac from the listed production sources (i.e., thorium cow or linac) to assess the reproducibility from lot to lot of ²²⁵Ac. The colors indicate the source of ²²⁵Ac used in the labeling process (blue (left) indicates thorium cow-derived ²²⁵Ac, and red (right) indicates linac-generated ²²⁵Ac). For each study, a single preparation of conjugated antibody was used, so the only variable in labeling was the source of ²²⁵Ac.

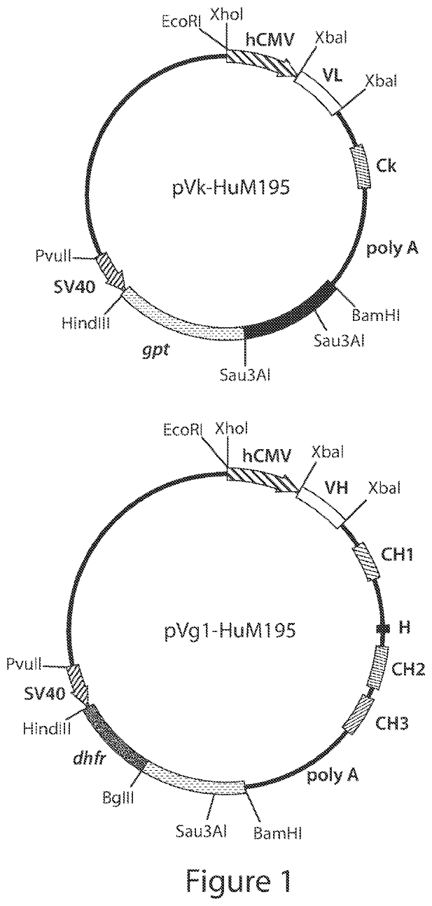

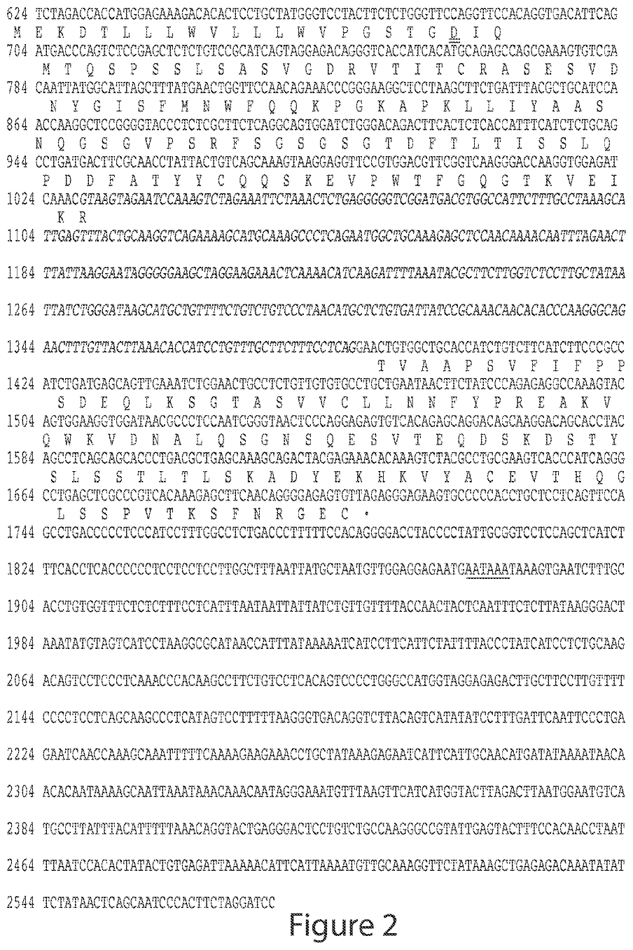

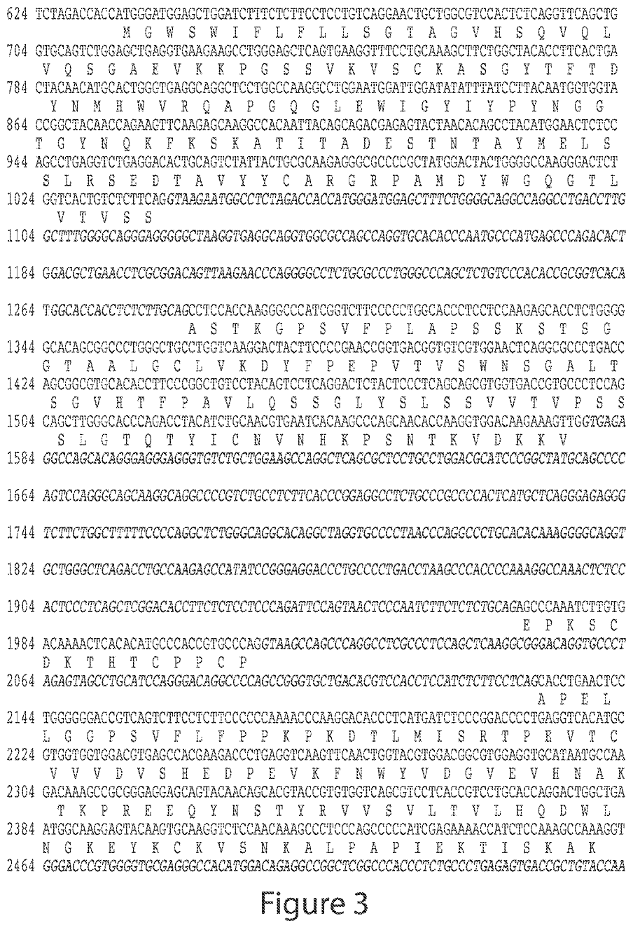

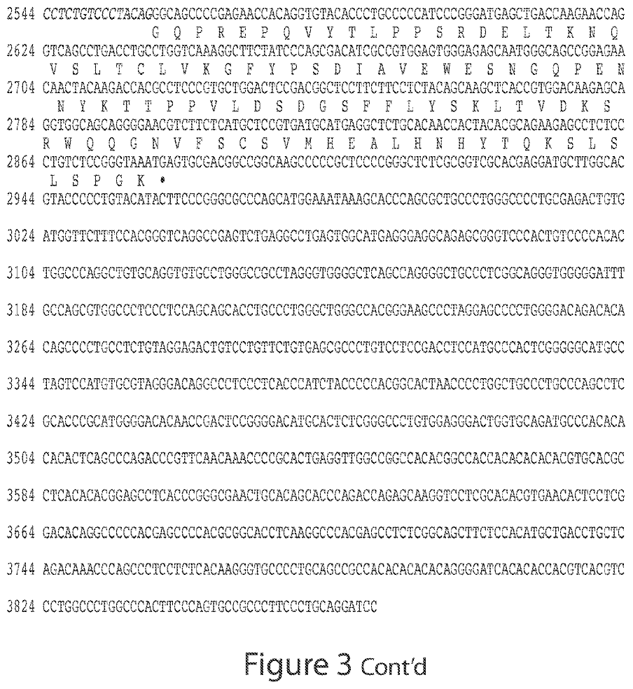

### DETAILED DESCRIPTION OF THE INVENTION

This invention provides a surprisingly effective method for producing ²²⁵Ac-conjugated therapeutic proteins, such as antibodies, using an isotopically mixed actinium preparation.

### Definitions

In this application, certain terms are used which shall have the meanings set forth as follows.

As used herein, “administer”, with respect to an agent (e.g., an actinium-labeled antibody), means to deliver the agent to a subject's body via any known method. Specific modes of administration include, without limitation, intravenous, oral, sublingual, transdermal, subcutaneous, intraperitoneal, intrathecal and intra-tumoral administration.

In addition, in this invention, the various agents (e.g., actinium-labeled antibodies) can be formulated using one or more routinely used pharmaceutically acceptable carriers. Such carriers are well known to those skilled in the art. For example, injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs and can contain excipients such as PLGA and polycaprylactone.

As used herein, the term “antibody” includes, without limitation, (a) an immunoglobulin molecule comprising two heavy chains and two light chains and which recognizes an antigen; (b) polyclonal and monoclonal immunoglobulin molecules; (c) monovalent and divalent fragments thereof (including peptide fragments), and (d) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies, and fragments thereof. Antibodies may be human, humanized or nonhuman. Antibodies include, for example, HuM195.

As used herein, an “anti-CD33 antibody” is an antibody that binds to any available epitope of CD33. In one embodiment, the anti-CD33 antibody binds to the epitope recognized by the antibody HuM195.

As used herein, a “chelator” can be any molecule capable of chelating an actinium atom and permitting its attachment to a therapeutic protein. Chelators and their methods of use are known, and include, without limitation, p-SCN-Bn-DOTA, and H₂macropa (Thiele, et al.).

As used herein, “conjugated”, with respect to a therapeutic protein and actinium atom, means bound, either covalently or non-covalently (e.g., via a chelator such as p-SCN-Bn-DOTA). The therapeutic protein, e.g., HuM195, can be bound to one or more of a plurality of actinium atoms, each atom being bound to a different amino acid residue. So, for example, a population of HuM195 antibodies conjugated using ^{225/7}Ac could include some antibodies bound to ²²⁵Ac but not to ²²⁷Ac, some antibodies bound to ²²⁷Ac but not to ²²⁵Ac, and some antibodies bound to both ²²⁷Ac and ²²⁵Ac. When the ^{225/7}Ac used for conjugating has a molar excess of ²²⁷Ac, that isotope would also be conjugated to the antibody population in excess of ²²⁵Ac. Conditions permitting conjugation (“conjugating conditions”) are known in the art, as discussed below.

A “hematologic malignancy”, also known as a blood cancer, is a cancer that originates in blood-forming tissue, such as the bone marrow or other cells of the immune system. Hematologic malignancies include, without limitation, leukemias (such as AML, acute promyelocytic leukemia, acute lymphoblastic leukemia, acute mixed lineage leukemia, chronic myeloid leukemia, chronic lymphocytic leukemia, hairy cell leukemia, large granular lymphocytic leukemia), myelodysplastic syndrome (MDS), myeloproliferative disorders (polycitermia vera, essential thrombocytosis, primary myelofibrosis and chronic myeloid leukemia), lymphomas, multiple myeloma, and MGUS and similar disorders.

As used herein, a “hematologic malignancy-associated antigen” can be, for example, a protein and/or carbohydrate marker found exclusively or predominantly on the surface of a cancer cell associated with that particular malignancy. Examples of hematologic malignancy-associated antigens include, without limitation, CD20, CD33, CD38, CD45, CD52, CD123 and CD319.

The antibody “HuM195” (also known as lintuzumab) is known, as are methods of making it. Likewise, methods of labeling HuM195 with ²²⁵Ac are known. These methods are exemplified, for example, in Scheinberg, et al. (U.S. Pat. No. 6,683,162) and Simon, et al. (U.S. Pat. No. 9,603,954). This information is also exemplified in the examples and figures below.

As used herein, the “molar ratio” of ²²⁷Ac to ²²⁵Ac means the ratio of the number of atoms of ²²⁷Ac to the number of atoms of ²²⁵Ac. This ratio differs dramatically from the ratio of radiation emission (e.g., alpha particle emission) between these two isotopes. For example, in a population of ^{225/7}Ac-labelled HuM195 wherein the molar ratio of ²²⁷Ac to ²²⁵Ac is five, the radiation ratio of ²²⁷Ac to ²²⁵Ac is below 0.01. In this invention, the molar ratio of ²²⁷Ac to ²²⁵Ac in each of the instant compositions and methods can be, for example: (i) 1:1, 2:1, 3:1, 4:1, 5:1, 6:1, 7:1, 8:1, 9:1 or 10:1; (ii) from 1:1 to 2:1, from 2:1 to 3:1, from 3:1 to 4:1, from 4:1 to 5:1, from 5:1 to 6:1, from 6:1 to 7:1, from 7:1 to 8:1, from 8:1 to 9:1, or from 9:1 to 10:1; (iii) from 5.0:1 to 5.1:1, from 5.1:1 to 5.2:1, from 5.2:1 to 5.3:1, from 5.3:1 to 5.4:1, from 5.4:1 to 5.5:1, from 5.5:1 to 5.6:1, from 5.6:1 to 5.7:1, from 5.7:1 to 5.8:1, from 5.8:1 to 5.9:1, or from 5.9:1 to 6.0:1; or (iv) 5.0:1, 5.05:1, 5.1:1, 5.15:1, 5.2:1, 5.25:1, 5.3:1, 5.35:1, 5.4:1, 5.45:1, 5.5:1, 5.55:1, 5.6:1, 5.65:1, 5.7:1, 5.75:1, 5.8:1, 5.85:1, 5.9:1, 5.95:1 or 6.0:1.

As used herein, a therapeutic protein “population” means a plurality of that therapeutic protein.

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Additionally, for a human subject afflicted with AML, the subject can be newly diagnosed, or relapsed and/or refractory, or in remission.

As used herein, a “therapeutic protein” has therapeutic value when conjugated to ²²⁵Ac. It may also have some therapeutic value in its unconjugated state, depending on the protein. Therapeutic proteins can be of any size and include, without limitation, therapeutic antibodies, therapeutic receptor derivatives and the like. Examples of therapeutic proteins include, without limitation, ²²⁵Ac-HuM195 and other antibody drugs that target CD33, as well as antibody drugs that target other hematologic malignancy-associated antigens. Further examples include ²²⁵Ac-daratumumab and other antibody drugs that target CD38, as well as the anti-PSMA drug ²²⁵Ac-PSMA-617 for treating prostate cancer.

Doses, i.e., “therapeutically effective amounts”, used in connection with this invention include, for example, a single administration, and two or more administrations (i.e., fractions). The amount administered in each dose can be measured, for example, by radiation (e.g., μCi/kg) or weight (e.g., mg/kg or mg/m²). In the case of ²²⁵Ac-HuM195 (also known as “Actimab-A”) for treating AML, dosing regimens include the following, without limitation: (i) 2×0.5 μCi/kg, 2×1.0 μCi/kg, 2×1.5 μCi/kg, or 2×2.0 μCi/kg, where the fractions are administered one week apart; (ii) 1×0.5 μCi/kg, 1×1.0 μCi/kg, 1×2.0 μCi/kg, 1×3.0 μCi/kg, or 1×4.0 μCi/kg; (iii) 1×15-20 μg/kg (0.03-0.06 μg/kg labeled); and (iv) less than or equal to approximately 2 mg per subject (approximately 0.04 mg labeled antibody per subject). Naturally, these doses can be adjusted accordingly to account for the presence of ²²⁷Ac-HuM195 in the subject compositions. In a preferred embodiment, the subject composition is administered (i) 1×, 2×, 4× or 8× per one-week period; (ii) 1×, 2×, 4× or 8× per two-week period; (i) 1×, 2×, 4× or 8× per three-week period; or (i) 1×, 2×, 4× or 8× per four-week period.

For an agent such as an antibody labeled with an alpha-emitting isotope, the majority of the drug administered to a subject typically consists of non-labeled antibody, with the minority being the labeled antibody.

As used herein, “treating” a subject afflicted with a disorder shall include, without limitation, (i) slowing, stopping or reversing the disorder's progression, (ii) slowing, stopping or reversing the progression of the disorder's symptoms, (iii) reducing the likelihood of the disorder's recurrence, and/or (iv) reducing the likelihood that the disorder's symptoms will recur. In the preferred embodiment, treating a subject afflicted with a disorder means (i) reversing the disorder's progression, ideally to the point of eliminating the disorder, and/or (ii) reversing the progression of the disorder's symptoms, ideally to the point of eliminating the symptoms and/or (iii) reducing or eliminating the likelihood of relapse (i.e., consolidation, which is a common goal of post remission therapy for AML and, ideally, results in the destruction of any remaining leukemia cells).

The treatment of a hematologic malignancy, such as AML, can be measured according to a number of clinical endpoints. These include, without limitation, survival time (such as weeks, months or years of improved survival time, e.g., one, two or more months of additional survival time), and response status (such as complete remission (CR), near complete remission (nCR), very good partial remission (VGPR) and partial remission (PR)).

In one embodiment, treatment of a hematologic malignancy, such as AML, can be measured in terms of remission. Included here are the following non-limiting examples. (1) Morphologic complete remission (“CR”): ANC≥1,000/mcl, platelet count ≥100,000/mcl, <5% bone marrow blasts, no Auer rods, no evidence of extramedullary disease. (No requirements for marrow cellularity, hemoglobin concentration). (2) Morphologic complete remission with incomplete blood count recovery (“CRi”): Same as CR but ANC may be <1,000/mcl and/or platelet count <100,000/mcl. (3) Partial remission (PR): ANC≥1,000/mcl, platelet count >100,000/mcl, and at least a 50% decrease in the percentage of marrow aspirate blasts to 5-25%, or marrow blasts <5% with persistent Auer rods. These criteria and others are known, and are described, for example, in SWOG Oncology Research Professional (ORP) Manual Volume I, Chapter 11A, Leukemia (2014).

### EMBODIMENTS OF THE INVENTION

The inventors have unexpectedly discovered that a mixture of ²²⁵Ac and a molar preponderance of ²²⁷Ac (“²²⁵Ac/²²⁷Ac preparation”, “²²⁵Ac/²²⁷Ac mixture”, “^{225/7}Ac preparation”, “^{225/7}Ac mixture”, or simply “^{225/7}Ac”) can be used to radioconjugate the anti-CD33 antibody HuM195 to produce a labeled drug having efficacy comparable to that of the counterpart drug labeled using pure ²²⁵Ac. ^{225/7}Ac can be obtained from high-energy accelerator bombardment of ²³²Th. This is significant, since ^{225/7}Ac can now serve as an alternative, and abundant, source for generating ²²⁵Ac-labelled biologics. Again, it is surprising that ^{225/7}Ac and pure ²²⁵Ac are equipotent for radio-conjugating protein-based drugs.

Specifically, this invention provides a first composition of matter comprising a therapeutic protein population wherein (a) each therapeutic protein in the population is conjugated to one or more actinium atoms, (b) each actinium atom is either ²²⁷Ac or ²²⁵Ac, and (c) the molar ratio of ²²⁷Ac to ²²⁵Ac in the composition is at least 1:1.

In a preferred embodiment, the first composition further comprises a molar excess of therapeutic protein not conjugated to any actinium atom. In this embodiment, the first composition comprises two sub-populations of the same protein (i.e., a first sub-population wherein each protein is conjugated to one or more actinium atoms, and a second sub-population wherein each protein is not conjugated to any actinium atom), wherein the molar ratio of the second sub-population to the first sub-population is greater than 1 (and ideally greater than 10, greater than 100, or greater than 1,000). That is, this invention provides a first composition of matter comprising (a) a first therapeutic protein sub-population wherein (i) each therapeutic protein in the first sub-population is conjugated to one or more actinium atoms, (ii) each actinium atom is either ²²⁷Ac or ²²⁵Ac, and (iii) the molar ratio of ²²⁷Ac to ²²⁵Ac in the composition is at least 1:1; and (b) a second therapeutic protein sub-population admixed with the first therapeutic protein sub-population, wherein each therapeutic protein in the second sub-population (which is the same protein as in the first sub-population) is not conjugated to an actinium atom, wherein the molar ratio of the second sub-population to the first sub-population is greater than 1 (and ideally greater than 10, greater than 100, or greater than 1,000).

In one embodiment, the molar ratio of ²²⁷Ac to ²²⁵Ac in the composition is between 5:1 and 6:1. In a preferred embodiment of the composition, the therapeutic protein is an antibody. Preferably, the antibody is HuM195 antibody. In another preferred embodiment of the composition, each actinium atom conjugated to a therapeutic protein is conjugated via a chelator. Preferably, the chelator is p-SCN-Bn-DOTA. In another preferred embodiment of the composition, the composition further comprises a pharmaceutically acceptable carrier (thereby constituting a first pharmaceutical composition).

This invention also provides a second composition of matter comprising a HuM195 antibody population wherein (a) each HuM195 antibody in the population is conjugated to one or more actinium atoms, (b) each conjugated actinium atom is conjugated via p-SCN-Bn-DOTA, (c) each actinium atom is either ²²⁷Ac or ²²⁵Ac, and (d) the molar ratio of ²²⁷Ac to ²²⁵Ac in the composition is between 5:1 and 6:1.

In a preferred embodiment, the second composition further comprises a molar excess of HuM195 antibody not conjugated to any actinium atom. In this embodiment, the second composition comprises two sub-populations of HuM195 antibody (i.e., a first sub-population wherein each HuM195 antibody is conjugated to one or more actinium atoms, and a second sub-population wherein each HuM195 antibody is not conjugated to any actinium atom), wherein the molar ratio of the second sub-population to the first sub-population is greater than 1 (and ideally greater than 10, greater than 100, or greater than 1,000). That is, this invention provides a second composition of matter comprising (a) a first HuM195 antibody sub-population wherein (i) each HuM195 antibody in the first sub-population is conjugated to one or more actinium atoms, (ii) each actinium atom is either ²²⁷Ac or ²²⁵Ac, and (iii) the molar ratio of ²²⁷Ac to ²²⁵Ac in the composition is at least 1:1; and (b) a second HuM195 antibody sub-population admixed with the first HuM195 antibody sub-population, wherein each HuM195 antibody in the second sub-population is not conjugated to an actinium atom, wherein the molar ratio of the second sub-population to the first sub-population is greater than 1 (and ideally greater than 10, greater than 100, or greater than 1,000).

In a preferred embodiment, the composition further comprises a pharmaceutically acceptable carrier (thereby constituting a second pharmaceutical composition).

This invention provides a third composition of matter comprising a population of chelated actinium atoms wherein (a) each actinium atom is either ²²⁷Ac or ²²⁵Ac, and (b) the molar ratio of ²²⁷Ac to ²²⁵Ac in the composition is at least 1:1. Preferably, this composition further comprises a molar excess of chelator. This composition is useful for conjugating an antibody drug, or example, with ²²⁵Ac.

In a preferred embodiment of the third composition, each chelated actinium atom comprises the actinium atom and p-SCN-Bn-DOTA. Preferably, the molar ratio of ²²⁷Ac to ²²⁵Ac in the third composition is between 5:1 and 6:1.

This invention further provides a fourth composition of matter comprising a population of chelated actinium atoms wherein (a) each actinium atom is either ²²⁷Ac or ²²⁵Ac, (b) each chelated actinium atom comprises the actinium atom and p-SCN-Bn-DOTA, and (c) the molar ratio of ²²⁷Ac to ²²⁵Ac in the composition is between 5:1 and 6:1.

This invention provides a first synthetic method for making a population of actinium-conjugated therapeutic proteins, comprising contacting, under conjugating conditions, (a) a population of therapeutic proteins and (b) a population of chelated actinium atoms wherein (i) each chelated actinium atom is either ²²⁷Ac or ²²⁵Ac, and (ii) the molar ratio of ²²⁷Ac to ²²⁵Ac in the population of chelated actinium atoms is at least 1:1.

In a preferred embodiment of the first synthetic method, the therapeutic protein is an antibody. Preferably, the antibody is HuM195 antibody. In another preferred embodiment of the first synthetic method, each chelated actinium atom comprises the actinium atom and p-SCN-Bn-DOTA. Preferably, antibodies are conjugated in the presence of an excess of chelator (e.g., p-SCN-Bn-DOTA), thereby making the chelator non-rate-limiting. Without being limited to any mechanistic theory, it is believed that this approach allows for ²²⁵Ac in the ²²⁵Ac/²²⁷Ac preparation to label a therapeutic antibody as efficiently as pure ²²⁵Ac obtained from a ²²⁹Th cow. Preferably, the molar ratio of ²²⁷Ac to ²²⁵Ac in the composition is between 5:1 and 6:1.

This invention provides a second synthetic method for making a population of actinium-conjugated HuM195 antibodies, comprising contacting, under conjugating conditions, (a) a population of HuM195 antibodies and (b) a population of actinium atoms chelated with p-SCN-Bn-DOTA, wherein (i) each chelated actinium atom is either ²²⁷Ac or ²²⁵Ac, and (ii) the molar ratio of ²²⁷Ac to ²²⁵Ac in the population of chelated actinium atoms is between 5:1 and 6:1.

This invention provides a first therapeutic method for treating a subject, preferably human, afflicted with a hematologic malignancy comprising administering to the subject a therapeutically effective amount of the first pharmaceutical composition, wherein the therapeutic protein is an anti-CD33 antibody.

In one embodiment of the first therapeutic method, the hematologic malignancy is acute myeloid leukemia, myelodysplastic syndrome (MDS) or multiple myeloma. Preferably, the hematologic malignancy is acute myeloid leukemia. In a preferred embodiment of the first therapeutic method, the anti-CD33 antibody is HuM195 antibody. In another preferred embodiment of the first therapeutic method, each actinium atom conjugated to a therapeutic protein is conjugated via a chelator. Preferably, the chelator is p-SCN-Bn-DOTA. In still another preferred embodiment of the first therapeutic method, the molar ratio of ²²⁷Ac to ²²⁵Ac in the composition is between 5:1 and 6:1.

This invention further provides a second therapeutic method for treating a subject, preferably human, afflicted with acute myeloid leukemia comprising administering to the subject a therapeutically effective amount of the second pharmaceutical composition.

This invention still further provides a composition of matter comprising (a) a pharmaceutically acceptable carrier, and (b) a population of chelated actinium atoms wherein (i) each chelated actinium atom is either ²²⁷Ac or ²²⁵Ac, and (ii) the molar ratio of ²²⁷Ac to ²²⁵Ac in the population of chelated actinium atoms is at least 1:1. Preferably, the molar ratio of ²²⁷Ac to ²²⁵Ac in the composition is between 5:1 and 6:1. Envisioned as part of this invention are methods for using this composition, for example, to (i) produce actinium-labeled therapeutic proteins, (ii) trace the metabolic or other fate of a molecule in vivo (i.e., serve as a tracer), or (iii) detect a fluid or chemical leak in an apparatus or other system.

In this invention, therapeutic small molecules may be employed, mutatis mutandis, as therapeutic proteins are employed.

This invention will be better understood by reference to the examples which follow, but those skilled in the art will readily appreciate that the specific examples detailed are only illustrative of the invention as described more fully in the claims which follow thereafter.

### EXAMPLES

### Example 1—Structure of ²²⁵Ac-Lintuzumab (²²⁵Ac-HuM195)

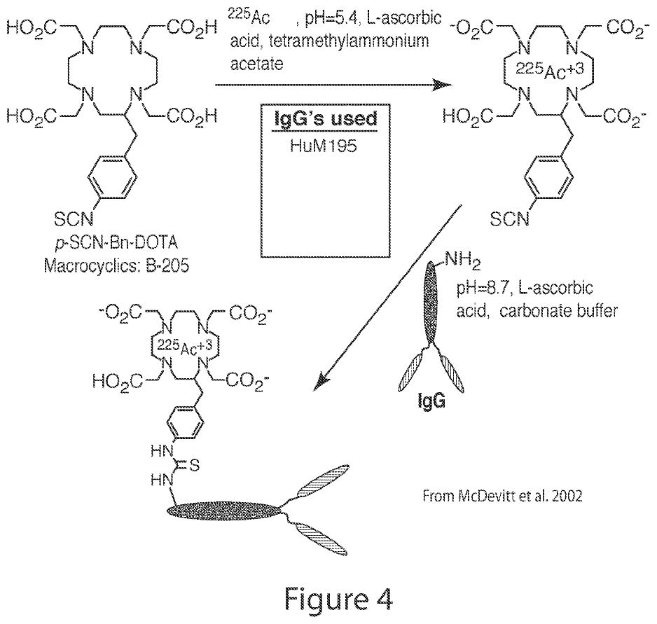

²²⁵Ac-Lintuzumab includes three key components; humanized monoclonal antibody HuM195 (generic name, lintuzumab), the alpha-emitting radioisotope ²²⁵Ac, and the bi-functional chelate (chelator) 2-(p-isothiocyanatobenzyl)-1,4,7,10-tetraazacyclododecane-1,4,7,10-tetraacetic acid (“p-SCN-Bn-DOTA”). As depicted in FIG. 4, HuM195 is radiolabeled using the bi-functional chelate p-SCN-Bn-DOTA that binds to ²²⁵Ac and that is covalently attached to the IgG via a lysine residue on the antibody.

### Example 2—p-SCN-Bn-DOTA

p-SCN-Bn-DOTA is 2-(4-Isothiocyanatobenzyl)-1,4,7,10-tetraazacyclododecane tetraacetic acid (Macrocyclics item code B205-GMP) and is synthesized by a multi-step organic synthesis that is fully described in U.S. Pat. No. 4,923,985.

### Example 3—Preparation of ²²⁵Ac-Lintuzumab (²²⁵Ac-HuM195)

One procedure for preparing ²²⁵Ac-Lintuzumab (2-step procedure) is based on the method described by Michael R. McDevitt (2002). The procedure involves radiolabeling the bi-functional chelate, p-SCN-Bn-DOTA, with the radioisotope ²²⁵Ac, followed by binding of the radiolabeled p-SCN-Bn-DOTA to the antibody (HuM195). The construct, ²²⁵Ac-p-SCN-Bn-DOTA-HuM195, is purified using 10 DG size exclusion chromatography and eluted with 1% human serum albumin (HSA). The resulting drug product, ²²⁵Ac-Lintuzumab, is then passed through a 0.2 μm sterilizing filter.

### Example 4—Process Flow for Preparation of ²²⁵Ac-Lintuzumab (²²⁵Ac-HuM195); Two-Step Process

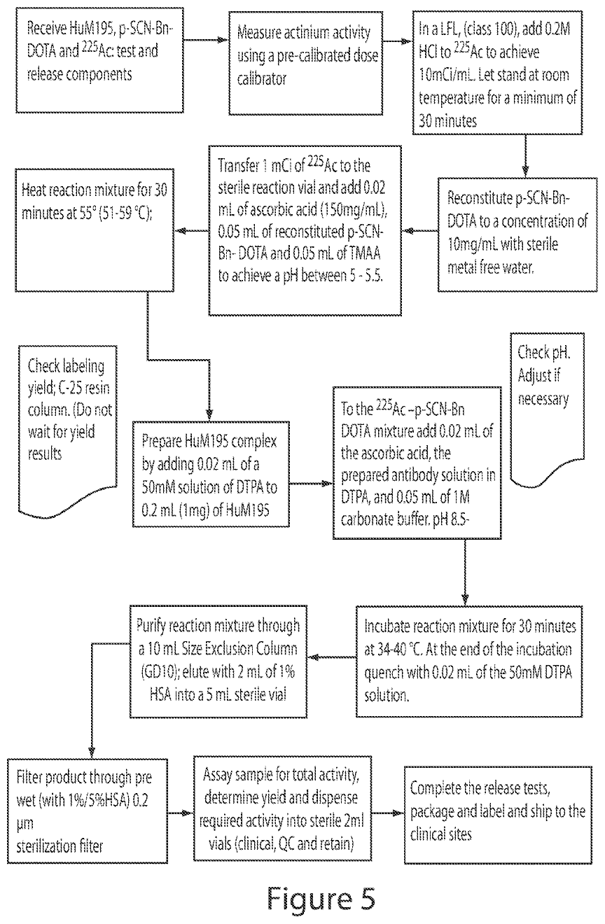

The two-step procedure, shown in FIG. 5, begins with confirming the identity of all components and the subsequent QC release of the components to production. The ²²⁵Ac is assayed to confirm the level of activity and is reconstituted to the desired activity concentration with hydrochloric acid. A vial of lyophilized p-SCNBn-DOTA is reconstituted with metal-free water to a concentration of 10 mg/mL. To the actinium reaction vial, 0.02 ml of ascorbic acid solution (150 mg/mL) and 0.05 ml of reconstituted p-SCN-Bn-DOTA are added and the pH adjusted to between 5 and 5.5 with 2M tetramethylammonium acetate (TMAA). The mixture is then heated at 55±4° C. for 30 minutes.

To determine the labeling efficiency of the ²²⁵Ac-p-SCN-Bn-DOTA, an aliquot of the reaction mixture is removed and applied to a 1 ml column of Sephadex C25 cation exchange resin. The product is eluted in 2-4 ml fractions with a 0.9% saline solution. The fraction of ²²⁵Ac activity that elutes is ²²⁵Ac-p-SCN-Bn-DOTA and the fraction that is retained on the column is un-chelated, unreactive ²²⁵Ac. Typically, the labeling efficiency is greater than 95%.

To the reaction mixture, 0.22 ml of previously prepared HuM195 in DTPA (1 mg HuM195) and 0.02 ml of ascorbic acid are added. The DTPA is added to bind any trace amounts of metals that may compete with the labeling of the antibody. The ascorbic acid is added as a radio-protectant. The pH is adjusted with carbonate buffer to pH 8.5-9. The mixture is heated at 37±3° C. for 30 minutes. The final product is purified by size exclusion chromatography using 10DG resin and eluted with 2 ml of 1% HSA. Typical reaction yields are 10%.

### Example 5—Process Flow for Preparation of ²²⁵Ac-Lintuzumab (²²⁵Ac-HuM195); One-Step Process

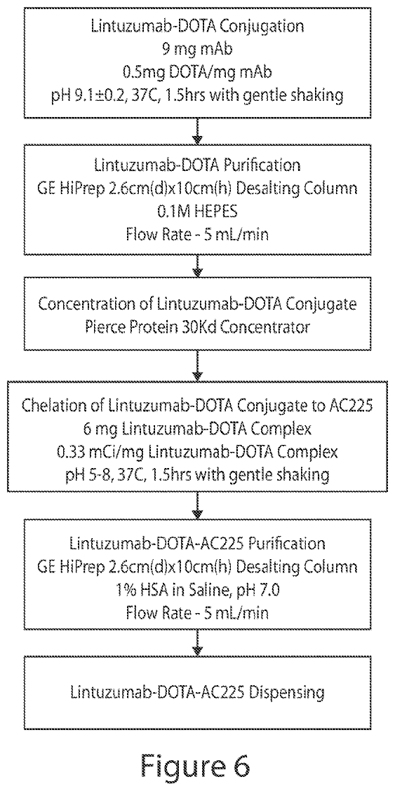

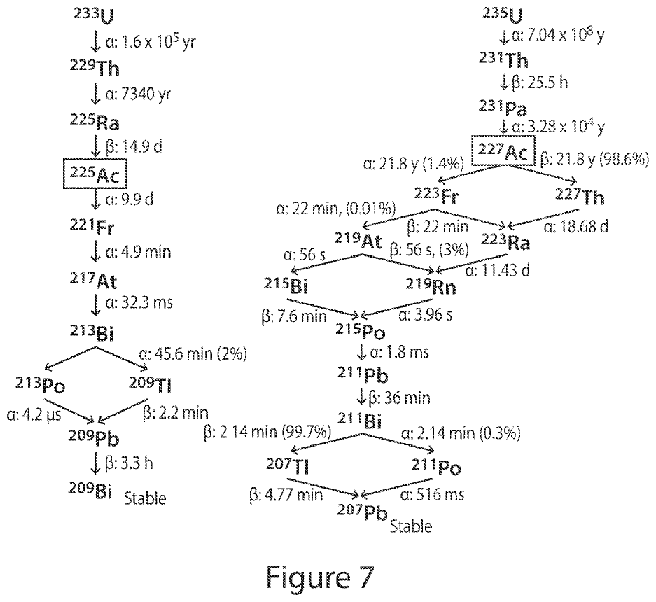

In this one-step procedure, shown in FIG. 6, a vial of lyophilized p-SCN-Bn-DOTA is reconstituted with metal-free water at a concentration of 10 mg/mL. To HuM195 antibody solution (5 mg/mL), p-SCN-Bn-DOTA is added at the ratio of 0.5 mg DOTA per mg of antibody and the pH of the reaction mixture is adjusted to 9.1±0.2 using 1M sodium bicarbonate. The reaction mixture is incubated at 37° C. for 1.5 hours with gentle shaking. Conjugate is purified using a HiPrep desalting column in 1 mL fractions. Fractions containing HuM195-DOTA conjugate are combined and concentrated using centrifuge filters with a 30 kDa molecular weight cutoff.

Actinium is dissolved using 0.2M hydrochloric acid at a concentration of 10 mCi/mL. Dissolved Ac225 is allowed to sit for 30 minutes before further processing. After incubation, an equal amount of 3M sodium acetate to hydrochloric acid is added to the actinium solution to adjust the pH between 5 and 8. To this solution, HuM195-DOTA is added at a ratio of 3 mg HuM195-DOTA per mCi of actinium. To this solution, ascorbic acid is added to adjust the pH of the reaction mixture between 6 and 7. The reaction mixture is incubated at 37° C. for 1.5 hours with gentle shaking. To quench unreacted metals in the solution, DTPA is added to the reaction mixture and the reaction is allowed to proceed for one more minute. The final product is purified using a HiPrep desalting column. Typical radiolabeling yields are about 60%-90%.

### Example 6—^{225/7}Ac-Labelling of HuM195

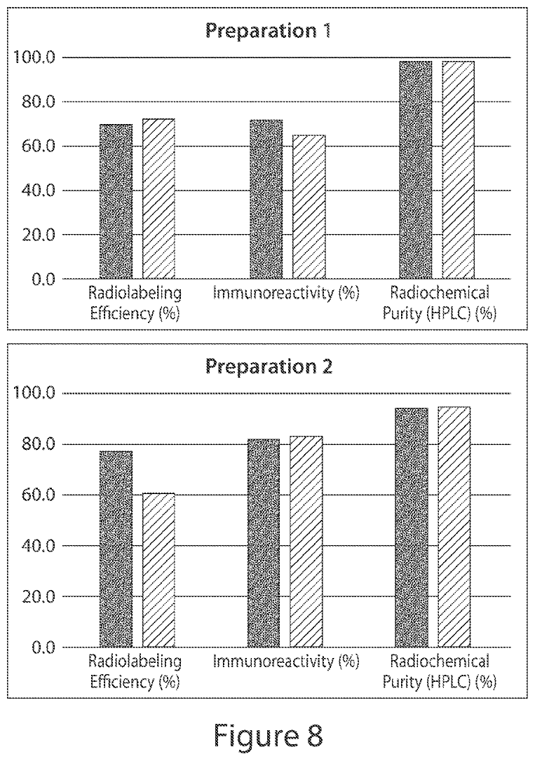

It is surprising that labeling HuM195 with DOTA-conjugated linac-generated ^{225/7}Ac under the same conditions used for labeling HuM195 with DOTA-conjugated ²²⁹Th cow-generated ²²⁵Ac (Simon) yielded a radioimmunoconjugate just as efficiently. It is also surprising that the two types of radioimmunoconjugates have similar immunoreactivity, radiochemical purity and potency (see Table 2 and FIG. 8).

Antibodies stably conjugated with DOTA (made as part of a 1-step process), such as through linkage with p-SCN-Bn-DOTA (Simon), typically contain multiple copies of p-SCN-Bn-DOTA linked to lysine amino acids present on the antibody. Since ^{225/7}Ac contains a mixture of free ²²⁵Ac and ²²⁷Ac, it would appear that the presence of more than one p-SCN-Bn-DOTA would be needed to provide sufficient sites for either a ²²⁵Ac or ²²⁷Ac to be chelated. Antibodies in this invention would have a range of 3-7 or as many as 8-16 stable p-SCN-Bn-DOTA linkages, depending on conjugation conditions (Molar ratio of DOTA to antibody: e.g., 10:1, or 100:1). With multiple p-SCN-Bn-DOTA linkages per antibody molecule within a conjugate preparation, p-SCN-Bn-DOTA chelator is presumably in excess relative to free ^{225/7}Ac even at a labeling concentration of 1:1 (e.g., 1mCi ^{225/7}Ac: 1 mg antibody). As shown in FIG. 8, 60-78% of all radioactive actinium is chelated, irrespective of ²²⁵Ac source. Since 99.3% or more of the radioactive energy is due to the high-energy ²²⁵Ac atoms, the results suggest that ²²⁵Ac is readily chelated and therefore is not outcompeted by ²²⁷Ac for chelation. In addition, the presence of ²²⁷Ac did not impair the immunoreactivity of the antibody. Thus wherein significant levels of ²²⁷Ac were likely chelated in the process, HuM195 antibody-DOTA conjugate was readily labeled with ^{225/7}Ac to high specific activity, without compromise of its ability to bind human CD33 antigen. Furthermore, functional testing of the potency of the radio-conjugates in vitro for tumor cell killing was performed. In this assay, tumor cells were incubated with titrations of each radio-conjugate for 60 minutes at 37 degrees. The cells were then washed three times to remove any unbound ²²⁵Ac-HuM195 radio-conjugate and incubated for up to four days for evidence of selective cell killing. In this assay, HuM195 conjugated with linac-generated ²²⁵Ac (i.e., ^{225/7}Ac) performed as well as HuM195 conjugated with ²²⁹Th cow-generated ²²⁵Ac in directing dose-dependent cell killing (data not shown).

TABLE 1
Ratio of ²²⁷Ac Atoms to ²²⁵Ac Atoms in Linac-Generated Actinium
Ratio of Ac-227 to Ac-225 atoms

| Activity of Ac 225 | 1 mCi |
|---|---|
| % of Ac-227 in sample | 0.700% |
| Ac-225 | Ac-227 |

| | | | | |
|---|---|---|---|---|
| Half life | | | 21.772 | years |
| | 10 | days | 7946.78 | days |
| | 240 | hours | 190722.7 | hours |
| | 14400 | min | 11443363 | min |
| Decay constant | 4.8135E−05 | 1/min | 6.06E−08 | 1/min |
| Activity | 1 | mCi | 0.007 | mCi |
| DPM | 2220000000 | dpm | 15540000 | dpm |
| # of atoms | 4.612E+13 | atoms | 2.57E+14 | atoms |
| moles | 2.2719E−10 | moles | 1.26E−09 | moles |

Ratio of Ac-227 to Ac-225 = 5.563
84.76% of the Mass of Ac is Ac-227

TABLE 2
HuM195-DOTA Conjugate: Labeling, Immunoreactivity and Purity

| | Preparation 1 | | Preparation 2 | |
|---|---|---|---|---|
| | NCMAcM195072718-A | NCMAcM195072718-B | NCMAcM195091418-A | NCMAcM195091418-B |
| | Thorium Cow Generated | Accelerator Generated | Thorium Cow Generated | Accelerator Generated |
| Radiolabeling Efficiency (%) | 69.6 | 72.5 | 77.2 | 60.2 |
| Immunoreactivity (%) | 72.2 | 65.4 | 82.0 | 83.0 |
| Radiochemical Purity (HPLC) (%) | 98.7 | 98.5 | 94.2 | 94.5 |

### Example 7—Specific Activity and HuM195 to Ac225 Ratios

Table 3 below shows specific activities of ²²⁵Ac per unit weight of HuM195 antibody, molar ratios of HuM195 antibody to ²²⁵Ac, and percentages of HuM195 antibody labeled with ²²⁵Ac. “Specific activity” means specific activity of Ac225 as milked (58,000 Ci/g); ²²⁵Ac molecular weight=225 g/mole; ²²⁵Ac activity per mole=13,050,000 Ci/mole; and molecular weight of HuM195=145,267 g/mole.

TABLE 3

| Specific | | | | |
|---|---|---|---|---|
| Activity (Ci/g or | Moles | Moles | Ratio | % mAb |
| mCi/mg mAb) | Actinium | mAb | mAb/Ac225 | Labeled |
| 0.05 | 3.83E−09 | 6.9E−06 | 1,797 | 0.06% |
| 0.1 | 7.66E−09 | 6.9E−06 | 898 | 0.11% |
| 0.2 | 1.53E−08 | 6.9E−06 | 449 | 0.22% |
| 0.3 | 2.30E−08 | 6.9E−06 | 299 | 0.33% |
| 0.4 | 3.07E−08 | 6.9E−06 | 225 | 0.45% |
| 0.5 | 3.83E−08 | 6.9E−06 | 180 | 0.56% |
| 0.6 | 4.60E−08 | 6.9E−06 | 150 | 0.67% |
| 0.7 | 5.36E−08 | 6.9E−06 | 128 | 0.78% |
| 0.8 | 6.13E−08 | 6.9E−06 | 112 | 0.89% |
| 0.9 | 6.90E−08 | 6.9E−06 | 100 | 1.00% |
| 1 | 7.66E−08 | 6.9E−06 | 90 | 1.11% |
| 1.1 | 8.43E−08 | 6.9E−06 | 82 | 1.22% |
| 1.2 | 9.20E−08 | 6.9E−06 | 75 | 1.34% |
| 1.3 | 9.96E−08 | 6.9E−06 | 69 | 1.45% |
| 1.4 | 1.07E−07 | 6.9E−06 | 64 | 1.56% |
| 1.5 | 1.15E−07 | 6.9E−06 | 60 | 1.67% |
| 1.6 | 1.23E−07 | 6.9E−06 | 56 | 1.78% |

### REFERENCES

1. Burnett, et al., “A Comparison of Low-Dose Cytarabine and Hydroxyurea With or Without All-trans Retinoic Acid for Acute Myeloid Leukemia and High-Risk Myelodysplastic Syndrome in Patients Not Considered Fit for Intensive Treatment”, Cancer. 2007; 109:1114-1124.
2. Co, et al., “Chimeric and humanized antibodies with specificity for the CD33 antigen.” J. Immunol. 1992; 148:1149-1154.
3. Dadachova, et al., “In vivo Evaluation of Free and Chelated Accelerator-produced Actinium-225-Radiation Dosimetry and Toxicity Results.” Curr Radiopharm. 2018; 11 (3): 215-222.
4. Domino and Baldor, “The 5-Minute Clinical Consult 2014.”
5. Gansow, et al., U.S. Pat. No. 4,923,985.
6. Harousseau, et al., “A randomized phase 3 study of tipifarnib compared with best supportive care, including hydroxyurea, in the treatment of newly diagnosed acute myeloid leukemia in patients 70 years or older”, Blood, 6 Aug. 2009, Vol. 114, No. 6.
7. Jurcic, et al., “Phase I Trial of Targeted Alpha-Particle Therapy with Actinium-225 (²²⁵Ac)-Lintuzumab and Low-Dose Cytarabine (LDAC) in Patients Age 60 or Older with Untreated Acute Myeloid Leukemia (AML)”, ASH 2016 Abstract.
8. Jurcic, J. “Clinical Studies with Bismuth-213 and Actinium-225 for Hematologic Malignancies.” Current Radiopharmaceuticals. 2018; 11:192-199.
9. Kratochwil, et al., “Targeted a-Therapy of Metastatic Castration-Resistant Prostate Cancer with ²²⁵Ac-PSMA-617: Dosimetry Estimate and Empiric Dose Finding.” J. Nuc. Med. 2017; 58:1624-1631.
10. Kumar, “Genetic Abnormalities and Challenges in the Treatment of Acute Myeloid Leukemia.” Genes & Cancer. 2011; 2:95-107.
11. Maguire, et al., “Efficient 1-Step Radiolabeling of Monoclonal Antibodies to High Specific Activity with ²²⁵Ac for a-Particle Radioimmunotherapy of Cancer.” J. Nuc. Med. 2014; 55:1492-1498.
12. McDevitt, et al., “Tumor therapy with targeted atomic nanogenerators.” Science. 2001; 294:1537-40.
13. McDevitt, “Design and synthesis of ²²⁵Ac radioimmunopharmaceuticals”, Applied Radiation and Isotope. 2002; 57:841-847.
14. Mulford et al., “The Promise of Targeted α-Particle Therapy”, The Journal of Nuclear Medicine, Vol. 46, No. 1 (Suppl), January 2005.
15. Mylotarg Wyeth Product Monograph (2005).
16. Nikula, et al., Alpha-emitting bismuth cyclohexylbenzyl DTPA constructs of recombinant humanized anti-CD33 antibodies: pharmacokinetics, bioactivity, toxicity and chemistry. J Nucl Med. 1999; 40:166-176.
17. Pollard, et al., “Correlation of CD33 expression level with disease characteristics and response to gemtuzumab ozogamicin containing chemotherapy in childhood AML”, Blood, 2012; 119:3705-11.
18. Radchenko, et al., “Application of ion exchange and extraction chromatography to the separation of actinium from proton-irradiated thorium metal for analytical purposes.” Journal of Chromatography. 2015; 1380:55-63.
19. Scheinberg, et al., U.S. Pat. No. 6,683,162.
20. Simon, et al., U.S. Pat. No. 9,603,954.
21. SWOG Oncology Research Professional (ORP) Manual, Volume I, Chapter 11A, Leukemia (2014).
22. Thiele, et al., “An Eighteen-Membered Macrocyclic Ligand for Actinium-225 Targeted Alpha Therapy”, Angew. Chem. Int. Ed. 2017, 56, 14712-14717.
23. V. H. J. van der Velden, et al., “High CD33-antigen loads in peripheral blood limit the efficacy of gemtuzumab ozogamicin (Mylotarg) treatment in acute myeloid leukemia patients”, Leukemia, 2004; 18:983-8.

24 Weidner, et al., “²²⁵Ac and 223Ra Production via 800 MeV Proton Irradiation of Natural Thorium Targets.” Appl Radiat Isot. 2012; 70(11):2590-2595.

1.-27. (canceled)

28. A method for making a population of actinium-conjugated antibodies, comprising:
(a) conjugating a chelator to a population of antibodies to produce chelator-conjugated antibodies, wherein the chelator is p-SCN-Bn-DOTA and wherein the molar ratio of p-SCN-Bn-DOTA to antibody is at least 3:1; and
(b) contacting the chelator-conjugated antibodies with a population of actinium atoms under chelating conditions to produce actinium-conjugated antibodies, wherein:
(i) each actinium atom is either 227Ac or 225Ac,
(ii) the molar ratio of 227Ac to 225Ac in the population of actinium atoms is between 4:1 and 7:1, and
(iii) the chelating conditions comprise a temperature of 37° C., a pH between 6 and 7, and an incubation time of at least 1 hour.

29. The method of claim 28, wherein the molar ratio of 227Ac to 225Ac in the population of actinium atoms is between 5:1 and 6:1.

30. The method of claim 28, wherein the molar ratio of p-SCN-Bn-DOTA to antibody in step (a) is between 3:1 and 16:1.

31. The method of claim 28, wherein the antibodies are HuM195 antibodies.

32. The method of claim 28, wherein the population of actinium atoms is produced by proton bombardment of a 232Th target.

33. The method of claim 28, wherein the chelating conditions in step (b) comprise an incubation time of between 1 hour and 2 hours.

34. The method of claim 28, wherein step (a) comprises:
(i) contacting the population of antibodies with p-SCN-Bn-DOTA at a ratio of 0.5 mg p-SCN-Bn-DOTA per mg of antibody;
(ii) adjusting the pH to between 8.5 and 9.5; and
(iii) incubating at 37° C. for at least 1 hour.

35. The method of claim 28, further comprising, after step (b), purifying the actinium-conjugated antibodies by size exclusion chromatography.

36. The method of claim 28, wherein at least 60% of the actinium atoms in the population of actinium atoms become conjugated to antibodies.

37. A composition of matter comprising a HuM195 antibody population wherein:
(a) at least 60% of the HuM195 antibodies in the population are each conjugated to one or more actinium atoms;
(b) each actinium atom conjugated to a HuM195 antibody is conjugated via p-SCN-Bn-DOTA;
(c) each conjugated actinium atom is either 227Ac or 225Ac; and
(d) the molar ratio of 227Ac to 225Ac in the conjugated actinium atoms is between 4.5:1 and 6.5:1.

38. The composition of claim 37, wherein the molar ratio of 227Ac to 225Ac is between 5:1 and 6:1.

39. The composition of claim 37, wherein each HuM195 antibody that is conjugated to one or more actinium atoms comprises between 3 and 16 molecules of p-SCN-Bn-DOTA.

40. The composition of claim 37, further comprising a pharmaceutically acceptable carrier.

41. The composition of claim 37, wherein the composition further comprises HuM195 antibodies not conjugated to any actinium atom, and wherein the molar ratio of unconjugated HuM195 antibodies to actinium-conjugated HuM195 antibodies is greater than 10:1.

42. The composition of claim 37, wherein the population of actinium atoms used to produce the composition was generated by proton bombardment of 232Th.

43. A method for treating a human subject afflicted with acute myeloid leukemia, comprising administering to the subject a therapeutically effective amount of a pharmaceutical composition comprising:
(a) a pharmaceutically acceptable carrier; and
(b) a HuM195 antibody population wherein:
(i) each HuM195 antibody in the population is conjugated via p-SCN-Bn-DOTA to one or more actinium atoms selected from 227Ac and 225Ac,
(ii) the molar ratio of 227Ac to 225Ac in the composition is between 4:1 and 7:1, and
(iii) the HuM195 antibody population retains at least 80% of the immunoreactivity of unconjugated HuM195 antibody as measured by binding to CD33 antigen.

44. The method of claim 43, wherein the molar ratio of 227Ac to 225Ac in the composition is between 5:1 and 6:1.

45. The method of claim 43, wherein the therapeutically effective amount comprises between 0.5 μCi/kg and 4.0 μCi/kg of 225Ac activity.

46. The method of claim 43, wherein the pharmaceutical composition is administered intravenously.

47. The method of claim 43, wherein the pharmaceutical composition is administered in two fractions of equal dose, wherein the fractions are administered one week apart.


SEQUENCE LISTING (4 sequences)

SEQ ID NO: 1 - DNA, 1952 bp, synthetic construct
  misc_feature 1..1952: note = Description of Artificial Sequence: Synthetic polynucleotide
  source 1..1952: mol_type = other DNA
tctagaccaccatggagaaagacacactcctgctatgggtcctacttctctgggttccaggttccacaggtgacattcagatgacccagtctccgagctctctgtccgcatcagtaggagacagggtcaccatcacatgcagagccagcgaaagtgtcgacaattatggcattagctttatgaactggttccaacagaaacccgggaaggctcctaagcttctgatttacgctgcatccaaccaaggctccggggtaccctctcgcttctcaggcagtggatctgggacagacttcactctcaccatttcatctctgcagcctgatgacttcgcaacctattactgtcagcaaagtaaggaggttccgtggacgttcggtcaagggaccaaggtggagatcaaacgtaagtagaatccaaagtctagaaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgctctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctaggatcc

SEQ ID NO: 2 - protein, 238 aa, synthetic construct
  REGION 1..238: note = Description of Artificial Sequence: Synthetic polypeptide
MEKDTLLLWVLLLWVPGSTGDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 3246 bp, synthetic construct
  misc_feature 1..3246: note = Description of Artificial Sequence: Synthetic polynucleotide
  source 1..3246: mol_type = other DNA
tctagaccaccatgggatggagctggatctttctcttcctcctgtcaggaactgctggcgtccactctcaggttcagctggtgcagtctggagctgaggtgaagaagcctgggagctcagtgaaggtttcctgcaaagcttctggctacaccttcactgactacaacatgcactgggtgaggcaggctcctggccaaggcctggaatggattggatatatttatccttacaatggtggtaccggctacaaccagaagttcaagagcaaggccacaattacagcagacgagagtactaacacagcctacatggaactctccagcctgaggtctgaggacactgcagtctattactgcgcaagagggcgccccgctatggactactggggccaagggactctggtcactgtctcttcaggtaagaatggcctctagaccaccatgggatggagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcacctgccctgggctgggccacgggaagccctaggagcccctggggacagacacacagcccctgcctctgtaggagactgtcctgttctgtgagcgccctgtcctccgacctccatgcccactcgggggcatgcctagtccatgtgcgtagggacaggccctccctcacccatctacccccacggcactaacccctggctgccctgcccagcctcgcacccgcatggggacacaaccgactccggggacatgcactctcgggccctgtggagggactggtgcagatgcccacacacacactcagcccagacccgttcaacaaaccccgcactgaggttggccggccacacggccaccacacacacacgtgcacgcctcacacacggagcctcacccgggcgaactgcacagcacccagaccagagcaaggtcctcgcacacgtgaacactcctcggacacaggcccccacgagccccacgcggcacctcaaggcccacgagcctctcggcagcttctccacatgctgacctgctcagacaaacccagccctcctctcacaagggtgcccctgcagccgccacacacacacaggggatcacacaccacgtcacgtccctggccctggcccacttcccagtgccgcccttccctgcaggatcc

SEQ ID NO: 4 - protein, 465 aa, synthetic construct
  REGION 1..465: note = Description of Artificial Sequence: Synthetic polypeptide
MGWSWIFLFLLSGTAGVHSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK